(19) United States
(12) Patent Application Publication
(10) Pub. No.: US 2008/0139966 A1
(43) Pub. Date: Jun. 12, 2008

(54) AUTOMATIC TONGUE DIAGNOSIS BASED ON CHROMATIC AND TEXTURAL FEATURES CLASSIFICATION USING BAYESIAN BELIEF NETWORKS

(75) Inventors: David ZHANG, Hong Kong (HK); Bo PANG, Hong Kong (HK)

Correspondence Address: BLAKELY SOKOLOFF TAYLOR & ZAFMAN, 1279 OAKMEAD PARKWAY, SUNNYVALE, CA, 94085-4040, US

(73) Assignee: THE HONG KONG POLYTECHNIC UNIVERSITY, Hong Kong (HK)

(21) Appl. No.: 11/608,243

(22) Filed: Dec. 7, 2006

(51) Int. Cl. A61B 5/00

(52) U.S. Cl. 600/590

(57) ABSTRACT

A tongue diagnosis system based on chromatic and textural features of a subject's tongue, which is based on Bayesian analysis of two sets of quantitative features, related to the color and texture of the tongue, respectively. The two sets of quantitative features are extracted from a digital tongue image of the tongue. The system includes several modules for image acquisition, tongue contour extraction, color and texture features extraction, and Bayesian analysis, respectively. These modules may be connected and configured in a way that a disease diagnosis process, from tongue image acquisition to an output of a diagnosis result, is progressing automatically.

### FIELD OF THE INVENTION

The present invention relates to disease diagnosis. More particularly, it relates to automatic disease diagnosis based on chromatic and textural features of the tongue using Bayesian Belief Networks.

### BACKGROUND OF THE INVENTION

Tongue diagnosis is an important diagnostic method in traditional Chinese medicine (TCM). Tongue diagnosis in traditional Chinese medicine has been described in literatures B. Kirschbaum, Atlas of Chinese Tongue Diagnosis, Eastland Press, July 2000, G. Maciocia, Tongue Diagnosis in Chinese Medicine, Eastland Press, June 1995, and N. M. Li, Chinese Tongue Diagnosis: A Comprehensive Reference, Shed-Yuan Publishing, 1994.

However, due to its qualitative, subjective and experience-based nature, traditional tongue diagnosis has a very limited application in modern medicine. Moreover, traditional tongue diagnosis is always concerned with the identification of syndromes (or patterns) rather than with the connection between tongue abnormal appearances and diseases. This is not well understood by practitioner of Western medicine, thus greatly obstructs its wider use in the world.

Recently, researchers have made considerable progress in standardization and quantification of tongue diagnosis. However, there are still significant problems with the existing approaches. First, some methods are only concerned with the identification of syndromes that are based on sophisticated yet esoteric terms in TCM; consequently they will not be widely accepted, especially in Western medicine. Second, the underlying validity of these methods and systems is usually based on a comparison between the diagnostic results that are obtained from the methods or systems and the judgments made by skillful practitioners of tongue diagnosis. In other words, subjectivity cannot be avoided when using such an approach. Last, many of the developed systems are only dedicated to the recognition of pathological features (such as the color of the tongue proper and the tongue coating) in tongue diagnosis, and the mapping from images of the tongue to diseases is not considered. This undoubtedly limits the applications of such systems in clinical medicine.

A Bayesian Brief Network (BBN), also known as Bayesian Network (BN), is a causal probabilistic network that compactly represents the joint probability distribution of a problem domain by exploiting conditional dependencies. BBN has been described in literatures J. Pearl, “Fusion, Propagation, and Structuring in Belief Networks,” Artificial Intelligence, Vol. 29, pp. 241-288, 1986, and N. Friedman, “Bayesian Network Classifiers,” Machine Learning, Vol. 29, pp. 131-163, 1997. Nowadays, with the help of powerful computers and new computational methods, Bayesian networks can be easily built and consequently has found applications in various areas. For example, Bayesian networks have been used for modeling knowledge in gene regulatory networks, medicine, engineering, text analysis, image processing, data fusion, and decision support systems.

The instant invention is believed to be the first time that a Bayesian network is employed in tongue diagnosis practice.

### SUMMARY OF THE INVENTION

According to one aspect of the present invention, there is provided a computerized tongue diagnosis method based on Bayesian belief networks (BBNs). It comprises the following steps: (a) taking a photograph of the tongue of a subject to be diagnosed which, if not already in a digital format, is converted to a digital image; (b) performing “Contour Extraction” where the pixels of the digital image are divided (and marked accordingly) into two groups: those within the tongue body and those out of the tongue body; (c) performing “Feature Extraction” where a set of values related to the color of the tongue body and a set of values related to the texture of the tongue body are extracted from the digital image; and (d) performing Bayesian analysis using the two sets of values as input to obtain a diagnostic result as the output. The modules performing the foregoing functions may be integrated into one or more physical devices so that the entire diagnosis process may be automated.

As another aspect of the present invention, there is provided a computerized system where the foregoing steps are performed consecutively and automatically in which the output of the previous step is automatically fed to the next step as the input. Using this automatic system, after taking a photograph of the tongue of the subject to be diagnosed, a diagnosis result may be obtained without further human intervention. The photograph can be taken by any image-capturing device, preferably, a device that produces digital images directly. The other steps can be carried out by software modules along with necessary hardware carriers.

The various features of novelty which characterize the invention are pointed out with particularity in the claims annexed to and forming a part of this disclosure. For a better understanding of the invention, its operating advantages, and specific objects attained by its use, reference should be made to the drawings and the following description in which there are illustrated and described preferred embodiments of the invention.

### BRIEF DESCRIPTION OF DRAWINGS

FIG. 1 outlines a tongue diagnosis system according to the present invention;

FIG. 2 shows the tongue image before (a) and after (b) Contour Extraction; and

FIG. 3 shows a structure of a joint belief network classifier (J-BNC) in an embodiment of tongue diagnosis system according to the present invention.

### DETAILED DESCRIPTION OF PARTICULAR EMBODIMENTS

Reference will now be made in detail to a particular embodiment of the invention as an example to facilitate the understanding of the present invention. Exemplary embodiments of the invention are described in detail, although it will be apparent to those skilled in the relevant art that some features that are not particularly important to an understanding of the invention may not be shown for the sake of clarity. On the other hand, details provided in connection with the particular embodiment are by example only, of which various changes and modifications may be effected by one skilled in the art without departing from the spirit or scope of the invention.

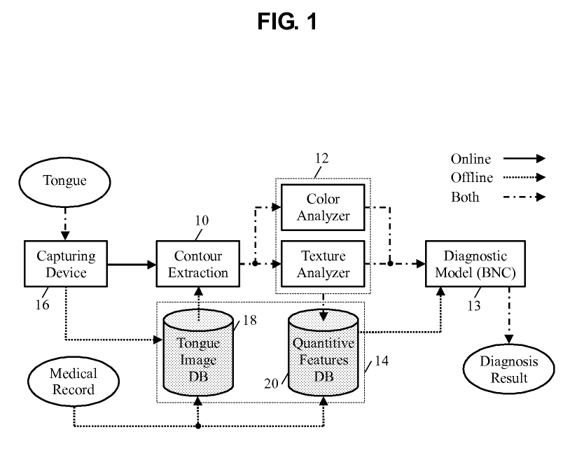

FIG. 1 outlines a particular tongue diagnosis system according to the present invention. The system has four modules: Contour Extraction Module 10, Feature Extraction Module 12 and Bayesian Network Classification Module 13, and optional Database Module 14. These modules may be implemented in software, hardware or combination of software and hardware. Some of the modules may not be necessary or built in another module.

Contour Extraction Module 10 is connected to an image acquisition or capturing device 16, such as, for example, an advanced 3-CCD camera, with a suitable lighting system. Of course, other types of capturing device may also be satisfactorily used, as long as the device can obtain a sufficiently high quality of true color photo images, which is essential to maintain the accuracy of diagnostic results. The photo images obtained from the capturing device 16, which is usually a rectangle image in a digital format and depicts the tongue as well as its neighboring parts, such as lips, teeth, etc, was fed as an input to the Contour Extraction Module 10, which can process all the pixels of photo image and distinguish the pixels showing the tongue (i.e., pixels within a contour of the tongue) from the pixels showing its neighbors (i.e., pixels outside the contour of the tongue) and mark all the pixels accordingly. Only are the pixels depicting the tongue body inputted to the next module, i.e., Feature Extraction Module 12. Of course, it is possible that the function of outputting only pixels within a contour of the tongue is integrated into the capturing device and therefore the Contour Extraction Module 10 would not be needed.

Feature Extraction Module 12 has two components: Color Analyzer and Texture Analyzer. The input was all the pixels within a contour of the tongue body from the Contour Extraction Module 10 and the output is a set of 32 values, 22 related to the color and 10 related to the texture of the tongue. Of course, it is possible to use more or less than the 32 values as used here. These 32 values are inputted to Bayesian Network Classification Module 13, which outputs a diagnosis result. The output of Feature Extraction Module 12 (i.e., the 32 values, in the present example) may also be fed into the quantitative features database 20 for the purpose of offline training and diagnosis. In addition to perform “online” diagnosis (meaning with a living subject), as shown in FIG. 1, the system may interact with additional modules, such as Database Modules 14, to perform “offline” diagnosis, that is, diagnosis based on medical records containing digital images of the tongue while the subject is not present. Before performing the actual diagnosis, Bayesian Network Classification Module had been trained and tested. The following further details each step.

### Contour Extraction of Tongue

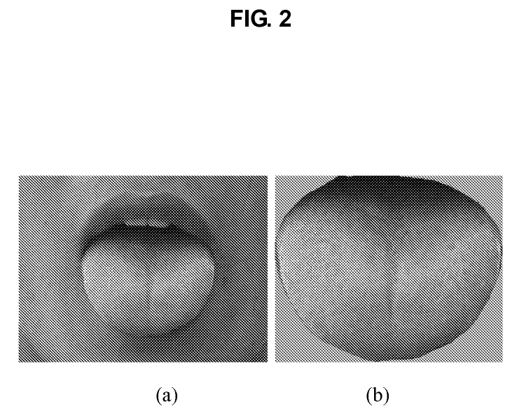

A tongue image sample obtained by the image acquisition or capturing device 16 is shown in FIG. 2(a). Alternatively, the system may retrieve images previous taken and stored in a file system or database 18. Before the processes of feature extraction and classification, an exact region that encompasses the surface of the tongue body was extracted from a tongue image, which usually includes the lips, part of the face, the teeth, etc. This was performed by Contour Extraction Module 10, internally using a combined model known as the bi-elliptical deformable contour (BEDC) to segment the tongue area from its surroundings. The output of the Contour Extraction Module 10 was 120 points on the tongue contour, which can be connected one by one to form a boundary or contour encompassing the surface of the tongue body in the image. The pixels within the boundary were marked as “in the tongue body” while the remaining pixels of the image were marked as “out of the tongue body.” Only the pixels in the tongue body were then inputted to the next module.

The details of BEDC, not forming part of this invention, has been disclosed in literatures B. Pang, K. Wang, D. Zhang, and F. Zhang, “On Automated Tongue Image Segmentation in Chinese Medicine,” Proceedings of 16^{th} International Conference on Pattern Recognition (ICPR' 2002), Vol. 1, pp. 616-619, August, 2002, and B. Pang, D. Zhang, and K. Wang, “The Bi-elliptical Deformable Contour and its Application to Automated Tongue Segmentation in Chinese Medicine,” IEEE Trans. on Medical Imaging, Vol. 24(8), pp. 946-956, 2005.

Briefly, an instance of BEDC is derived from a bi-elliptical parameterization of the deformable template (called BEDT), which refers to a structure composed of two semi-ellipses with a common center. The main purpose of the BEDT is to increase the robustness of the algorithm to noises that are usually caused by pathological details in this case. By applying the BEDT, a rough segmentation can be obtained through an optimization process in the model's parameter space using a gradient descent method. Then, the BEDT may be sampled to form a deformable contour (i.e. the BEDC). To further improve the performance, an elliptical template force may be introduced into the BEDC to replace the traditional internal force, which is capable of accurate local control. An example of the segmented tongue area using the BEDC is shown in FIG. 2(b).

### Quantitative Feature Extraction

Pathological features appearing in traditional tongue diagnosis theories (see G. Maciocia, Tongue Diagnosis in Chinese Medicine, Eastland Press, June 1995) are all qualitative, thus subjective, using descriptions such as “reddish purple tongue”, “white, thin and slippery coating”, and so on. Based on the understanding that many descriptive features in traditional tongue diagnosis indicate some implicit relations to color and texture related features, such as “reddish purple”, “white”, “thin”, “slippery”, to name just a few, this embodiment employed several general chromatic and textural measurements (see I. Pita, “Fundamentals of Color Image Processing”, in: Digital Image Processing Algorithms, Prentice-Hall, Englewood Cliffs, N.J., pp. 23-40, 1993, and T. R. Reed and J. M. H. DuBuff, “A Review of Recent Texture Segmentation and Feature Extraction Techniques,” CVGIP: Image Understanding, Vol. 57, No. 3, pp. 359-372, May 1993) and took no considerations of whether these measurements could well correspond to specific qualitative features used in traditional tongue diagnosis. Nevertheless, a diagnostically useful subset of these quantitative features was discovered through an integrated feature-selection procedure in the training algorithm of Bayesian networks and used as basis for tongue diagnosis in the present invention. These quantitative features about color and texture are detailed in the following.

### Chromatic Features:

A color is to be given in relation to a specific color space, and the extraction of color features can be performed in different color spaces, which usually includes RGB, HSV, CIEYxy, CIELUV and CIELAB. Different from other color spaces, the HSV color space is an intuitive system in which a specific color is described by its hue, saturation and brightness values, it has discontinuities in the value of the hue around red, which make this approach noise-sensitive. Therefore, the other four color spaces (i.e., RGB, CIEYxy, CIELUV and CIELAB) were used for extraction of quantitative color features.

The color-related measurements that were used in this embodiment are the means and standard deviations of values of effective pixels measured in each color plane of all the four color spaces, each having three planes. Thus, there were a total of 22 different color-related measures: CRᵢ (i=1,2,Λ,11), which represents means of values of effective pixels measured in each color plane of the four color spaces, and CRⱼ(j=12,13,Λ,22), which represents standard deviations of values of effective pixels measured in each color plane of the four color spaces. Since the L channels (or planes) in CIELUV and CIELAB spaces both represent the sensation of the lightness in the human vision system, only one of them was used. “Effective pixels” here means all the pixels within the tongue region, i.e., what were outputted from contour extraction module. It is within ordinary skill in the art to calculate means and standard deviations of values of effective pixels measured in each color plane of all the four color spaces.

### Textural Features:

Two feature-based texture operators, both derived from the same co-occurrence matrix, were used to extract different textural features from images of the tongue. These two operators are the second moment and the contrast measures based on a co-occurrence matrix, which are shown as follows:

$${W}_{M} = {\sum }_{{g}_{1}} {\sum }_{{g}_{2}} {P}^{2} ( {g}_{1} , {g}_{2} )$$

$${W}_{C} = {\sum }_{{g}_{1}} {\sum }_{{g}_{2}} \vert {g}_{1} - {g}_{2} \vert P ( {g}_{1} , {g}_{2} )$$

where P(g₁, g₂) is a co-occurrence matrix and g₁ and g₂ are two values of the gray level. W_{M} measures the smoothness or homogeneity of an image and it reaches its minimum value when all of the P(g₁, g₂) have the same value. W_{C} is the first moment of the differences in the values of the gray level between the entries in a co-occurrence matrix. Both of the two textural descriptors are calculated quantitatively and they have little correlation with the sensation of the human vision system.

Based on the theory in Traditional Chinese Medicine that there is a mapping between various internal organs and different regions of the tongue, (see N. M. Li, Y. F. Wang, Z. C. Li, et al., Diagnosis through Inspection of the Tongue, Heilongjiang Science and Technology Press, March 1987), the tongue was partitioned into five regions and the above two textural measures for each partition of the tongue were calculated. For convenience, each partition of a tongue is denoted with a digit: 1: Tip of the tongue; 2: Left edge of the tongue; 3: Center of the tongue; 4: Right edge of the tongue; and 5: Root of the tongue. Thus, a set of textural measurements for each tongue were obtained, containing a total of 10 texture measures as follows:

$$\{ \begin{matrix}{TR}_{i} = {W}_{M , i} \\ {TR}_{i + 5} = {W}_{C , i}\end{matrix} ( i = 1 , 2 , Λ , 5 ) , \qquad ( 2 )$$

where W_{M,i} and W_{C,i} denote the measurements of W_{M} and W_{C} for partition i, respectively. It is within ordinary skill in the art to calculate the 10 texture-related values based on the above equations and descriptions.

### Tongue Diagnosis Using Bayesian Networks

Although Bayesian belief networks provide a natural and efficient representation to encode prior knowledge, this particular embodiment of the present invention did not employ any such information when constructing a diagnostic model, i.e., the BNC model built during the training process. Consequently, both the graphical structure and the conditional probability tables of the BNC were learned using statistical algorithms from data, which is built in Bayesian Network PowerPredictor, a software program which was used to train and test a Bayesian network classifier. The Bayesian Network PowerPredictor, developed by J. Cheng, is freely available by downloading from his website, (see PowerPredictor System, http://www.cs.ualberta.ca/˜jcheng/bnpp.htm). Within the present application, the following terms are used interchangeably: “Bayesian Network,” “Belief Network,” and “Bayesian Belief Network.”

In this particular embodiment, an Access database file (the mdb file) was used to train BNCs by using the Bayesian Network PowerPredictor. In the mdb file, each row contains the information of a particular subject: the first column (field) is the reference to a sign of a disease; the second column is the ID of the image specimen of the particular subject; columns 3-24 contain the 22 color measurements extracted from the specimen, respectively; and columns 25-34 contain the 10 texture measurements extracted from the specimen, respectively. After training, the Bayesian Network PowerPredictor produced a BNC file, which records the parameters and structure of the trained Bayesian Network and is used internally by the Bayesian Network PowerPredictor. An mdb file having the same structure as the one used for training was used for testing on or actual performing diagnosis with the trained Bayesian Network. For performing diagnosis, however, only the subset of relevant features selected during the training process is used. The BNC file stores the information specifying the subset of relevant features. For an actual diagnosis, the Bayesian Network PowerPredictor takes a database entry (i.e., all the relevant data in a row) and produces a set of probabilities, each of which indicates how likely the specimen belongs to a specific diagnostic category. In this particular embodiment of the present patent, there were 14 diagnostic categories (13 diseases and 1 healthy), so the actual output of a BNC was a set of 14 probabilities. Here, the disease ID corresponding to the highest probability was taken as the diagnosing result. However, the BNC output may be used differently. For example, one can take three IDs corresponding to the highest three probabilities, as the candidate resulting set of a diagnosis. Of course, it is possible that the BNC output may include fewer or more than 14 diagnostic categories in other embodiments designed by people skilled in the art.

### Diagnosis Results

A total of 525 subjects, including 455 patients and 70 healthy volunteers, were involved in the experiments. There were totally 13 common internal diseases included (see Table 1). The patients were all in-patients mainly from five different departments at the No. 211 Harbin Hospital, and the healthy volunteers were chosen from the campus students of Harbin Institute of Technology. A total of 525 digital tongue images were taken, exactly one for each subject, as the experimental samples.

A stratified 10-fold cross-validation technique was utilized in all of the following experiments to evaluate all the classifiers. 10-fold cross-validation (CV) technique partitions a pool of labeled data, S, into 10 approximately equally-sized subsets. Each subset was used as a test set for a classifier trained on the remaining 9 subsets. The empirical accuracy was given by the average of the accuracies of these 10 classifiers. When employing a stratified partitioning in which the subsets contain approximately the same proportion of classes as S, a stratified 10-fold cross-validation was obtained, which can reduce the estimate's variance.

In the first experiment, a belief network classifier (BNC) based on textural features, called a texture BNC (T-BNC), was trained using only texture features extracted from all samples in the training set. The diagnostic results are shown in Table 2. As shown, the average true positive rate (TPR) is about 26.1%, which suggests that textural features utilized in this study are not sufficiently discriminating for diagnosing the diseases. Nevertheless, employing textural features in some diagnoses, such as appendicitis (D03), pancreatitis (D04), and coronary heart disease (D10), are shown to be more meaningful.

On the other hand, the performance of chromatic features (used in a color BNC, or C-BNC) in the diagnoses of these internal diseases is significantly better compared to that of textural features, which is up to 62.3%. It should be noticed that diagnosis of pancreatitis scores best, reflecting the fact that a patient with pancreatitis usually has a distinct bluish tongue.

Finally, when both chromatic and textural features were used to construct a joint BNC (J-BNC) for the classification of these diseases, the average TPR is about 75.8%, and for three diseases (appendicitis, pancreatitis, and coronary heart disease) the TPRs are even higher than 85%.

TABLE 1
List of the 13 internal diseases and healthy subjects

| Disease ID | Disease | Number of files |
|---|---|---|
| D00 | Healthy | 70 |
| D01 | Intestinal infarction | 11 |
| D02 | Cholecystitis | 21 |
| D03 | Appendicitis | 43 |
| D04 | Pancreatitis | 41 |
| D05 | Nephritis | 17 |
| D06 | Diabetes mellitus | 49 |
| D07 | Hypertension | 65 |
| D08 | Heart failure | 17 |
| D09 | Pulmonary heart disease | 21 |
| D10 | Coronary heart disease | 71 |
| D11 | Hepatocirrhosis | 25 |
| D12 | Cerebral infarction | 30 |
| D13 | Upper respiratory | 44 |
| | infection | |

TABLE 2
Diagnostic results (TPR) of various belief network classifiers (BNC).

| Disease ID | T-BNC | C-BNC | J-BNC |
|---|---|---|---|
| D00 | 20.0 | 50.0 | 77.1 |
| D01 | 9.1 | 45.5 | 63.6 |
| D02 | 4.8 | 42.9 | 61.9 |
| D03 | 53.5 | 86.0 | 93.0 |
| D04 | 70.7 | 90.2 | 100 |
| D05 | 5.9 | 17.6 | 23.5 |
| D06 | 4.1 | 53.1 | 65.3 |
| D07 | 3.1 | 61.5 | 75.4 |
| D08 | 5.9 | 35.3 | 35.3 |
| D09 | 4.8 | 47.6 | 71.4 |
| D10 | 64.8 | 90.1 | 93.0 |
| D11 | 12 | 48.0 | 64.0 |
| D12 | 13.3 | 60.0 | 80.0 |
| D13 | 20.5 | 56.8 | 70.5 |
| Average | 26.1 | 62.3 | 75.8 |

TABLE 3
Definition of CR₁–CR₂₂.

| | |
|---|---|
| Means | Standard Deviations |

| | | | |
|---|---|---|---|
| CR₁ | R (in RGB) | CR₁₂ | R (in RGB) |
| CR₂ | G (in RGB) | CR₁₃ | G (in RGB) |
| CR₃ | B (in RGB) | CR₁₄ | B (in RGB) |
| CR₄ | Y (in CIEYxy) | CR₁₅ | Y (in CIEYxy) |
| CR₅ | x (in CIEYxy) | CR₁₆ | x (in CIEYxy) |
| CR₆ | y (in CIEYxy) | CR₁₇ | y (in CIEYxy) |
| CR₇ | L (in CIELUV) | CR₁₈ | L (in CIELUV) |
| CR₈ | U (in CIELUV) | CR₁₉ | U (in CIELUV) |
| CR₉ | V (in CIELUV) | CR₂₀ | V (in CIELUV) |
| CR₁₀ | A (in CIELAB) | CR₂₁ | A (in CIELAB) |
| CR₁₁ | B (in CIELAb) | CR₂₂ | B (in CIELAb) |

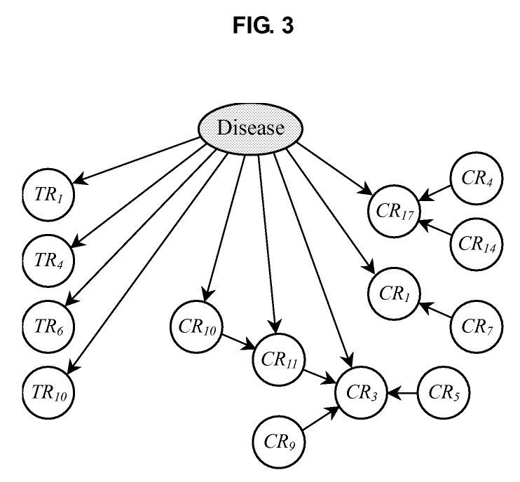

The graphical structure of the J-BNC is illustrated in FIG. 3. The definitions for CR₁-CR₂₂ are provided in Table 3. An internal feature selection process of the training algorithm utilized in the Bayesian Network PowerPredictor software finally identified 4 textural features and 10 chromatic features, out of original 32 quantitative features, for the classification and produced the graphical structure shown in FIG. 3. Among those 4 textural features, the two corresponding to the tip of the tongue, namely TR₁ and TR₆, are selected as feature nodes, which demonstrates that from the statistic point of view, textural features of the tip of the tongue are most discriminating for the diagnosis of the these diseases. Similarly, chromatic features related to the means of the aforementioned 4 color spaces have more significance for the classification, since 8 of the 10 surviving chromatic features of the final J-BNC are means-connected.

As the above results demonstrate, mapping from quantitative features of the tongue (including chromatic and textural features) to diseases in human subjects using a Bayesian network provides valuable tool for disease diagnosis.

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims.

What is claimed is:

1. A method for diagnosing disease, comprising the steps of:
(a) acquiring a digital image of a subject's tongue;
(b) extracting from said digital image a first plurality of data relating to color of the tongue and a second plurality of data relating to texture of the tongue; and
(c) performing a Bayesian analysis based on a trained Bayesian network, which uses said first plurality of data and second plurality of data as input and outputting a diagnosis result.

2. The method of claim 1, wherein said first plurality of data comprises means of one or more color planes in one or more color spaces.

3. The method of claim 2, wherein said first plurality of data further comprises standard deviations of one or more color planes in one or more color spaces.

4. The method of claim 3, wherein said second plurality of data comprises W_{M} and W_{C} in one or more tongue partitions in said digital image, W_{M} being a measurement of smoothness or homogeneity of a partition and W_{C} being a measurement of the first moment of the differences in the values of the gray level between the entries in a co-occurrence matrix.

5. The method of claim 4, wherein W_{M} and W_{C} are calculated based on the following equations:
$${W}_{M} = {\sum }_{{g}_{1}} {\sum }_{{g}_{2}} {P}^{2} ( {g}_{1} , {g}_{2} )$$
$${W}_{C} = {\sum }_{{g}_{1}} {\sum }_{{g}_{2}} \vert {g}_{1} - {g}_{2} \vert P ( {g}_{1} , {g}_{2} ) ,$$
where P(g₁, g₂) is a co-occurrence matrix and g₁ and g₂ are two values of the gray level.

6. The method of claim 5, wherein said W_{M} and W_{C} are calculated in one or more tongue partitions selected from the group consisting of: tip of the tongue, left edge of the tongue, center of the tongue, right edge of the tongue, and root of the tongue.

7. The method of claim 6, wherein said Bayesian analysis is performed using a computer software program Bayesian Network PowerPredictor.

8. The method of claim 1, wherein step (a) comprises taking a photograph of the tongue with a capturing device and marking or extracting pixels within a contour encompassing the body of the tongue.

9. A system for diagnosing disease in a human subject, comprising the following elements:
(a) a module for obtaining or storing an image of the tongue;
(b) a module for marking or extracting pixels within a contour encompassing the body of the tongue from said image;
(c) a module for extracting a plurality of data relating to color of the tongue or data relating to texture of the tongue; and
(d) a module for performing a Bayesian analysis using said plurality of data from said module (c) as input to produce a diagnosis result;
wherein said modules (a) to (d) are implemented in software, hardware or combination of software and hardware.

10. The system of claim 9, wherein said module (a) is a digital camera or video camera and said module (b) is internal or external to said module (a).

11. The system of claim 9, wherein said module (a) is connected to module (b) and outputs an image, which is inputted to module (b).

12. The system of claim 9, wherein said module (b) is connected to module (c) and produces an output, which is inputted to module (c).

13. The system of claim 12, wherein said module (c) is connected to module (d) and produces an output, which is inputted to module (d).

14. The system of claim 9, wherein said module (a) in connected to said module (b), which is connected to said module (c), which is connected to said module (d), and where upon acquiring an image of the tongue of a subject, a disease diagnosis process proceeds automatically without human intervention up to resulting in a diagnosis result.

15. The system of claim 9, wherein said module (c) is configured or programmed to perform calculations according to the following equations:
$${W}_{M} = {\sum }_{{g}_{1}} {\sum }_{{g}_{2}} {P}^{2} ( {g}_{1} , {g}_{2} )$$
$${W}_{C} = {\sum }_{{g}_{1}} {\sum }_{{g}_{2}} \vert {g}_{1} - {g}_{2} \vert P ( {g}_{1} , {g}_{2} ) ,$$
where P(g₁, g₂) is a co-occurrence matrix and g₁ and g₂ are two values of the gray level.

16. The system of claim 15, wherein said module(c) is configured or programmed to further perform calculations to obtain means and standard deviations of a plurality of pixels of a digital image, measured in one or more color planes in one or more color spaces.

17. The system of claim 9, wherein said module (d) is a computer software program Bayesian Network PowerPredictor.

18. The system of claim 9, wherein said module (b) uses a bi-elliptical deformable contour model to separate the tongue area from its surroundings in said image of the tongue.
